(19) United States
(12) Patent Application Publication
(10) Pub. No.: US 2020/0398003 A1
(43) Pub. Date: Dec. 24, 2020

(54) MELTING DEVICE AND MELTING METHOD

(71) Applicant: TERUMO KABUSHIKI KAISHA, Tokyo (JP)

(72) Inventor: YUSUKE NOZAKI, Kanagawa (JP)

(21) Appl. No.: 17/014,713

(22) Filed: Sep. 8, 2020

Related U.S. Application Data
(63) Continuation of application No. PCT/JP2019/009319, filed on Mar. 8, 2019.

(30) Foreign Application Priority Data
Mar. 13, 2018 (JP) 2018-045050

(51) Int. Cl. A61M 5/44; A01N 1/02; A61K 35/12

(52) U.S. Cl. CPC A61M 5/445; A01N 1/0263; A61M 2205/3606; A61K 35/12; A61M 2205/366; A01N 1/0242

(57) ABSTRACT

A melting device is provided that melts a bio-derived frozen product contained in a container. The melting device includes a main body capable of supporting the container in a vertically oriented state and a heating unit that heats only an upper portion of the container supported in the vertically oriented state. A liquid (bio-derived liquid product) obtained as the bio-derived frozen product melts in the upper portion of the container and then flows to a lower portion of the container. The bio-derived frozen product that has not been melted in the container floats in the liquid, and thus, is heated in the upper portion of the container to be melted.

### CROSS REFERENCE TO RELATED APPLICATIONS

The present application is a continuation of and claims benefit to PCT Application No. PCT/JP2019/009319 filed on Mar. 8, 2019, entitled “MELTING DEVICE AND MELTING METHOD” which claims priority to Japanese Patent Application No. 2018-045050 filed on Mar. 13, 2018. The entire disclosures of the applications listed above are hereby incorporated herein by reference, in their entirety, for all that they teach and for all purposes.

### FIELD

The present disclosure relates to a melting device and a melting method for melting a bio-derived frozen product contained in a container.

### BACKGROUND

Conventionally, a method of immersing a container in a constant temperature water tank is generally used to melt (e.g., thaw, unfreeze, etc.) a bio-derived frozen product contained in the container such as a bag. However, there is a concern regarding a hygienic problem in such a melting method using hot water. European Patent Application No. 0318924 proposes a device that sandwiches a container from above and below with two heating bags and melts a bio-derived frozen product in the container.

### SUMMARY

In the related art, however, the heat transfer from the heating bag to the container is not efficiently performed so it takes a long time to melt the bio-derived frozen product.

The present disclosure has been made in consideration of such problems, and an object thereof is to provide a melting device and a melting method capable of hygienically and quickly melting a bio-derived frozen product contained in a container.

In order to achieve the above object, one aspect of the present disclosure is a melting device that melts a bio-derived frozen product contained in a container, and includes: a main body capable of supporting the container in a vertically oriented state; and a heating unit that heats only an upper portion of the container supported in the vertically oriented state.

According to this melting device, the liquid in which the bio-derived frozen product is melted at the upper portion of the container (bio-derived liquid product) flows to the lower portion of the container. The bio-derived frozen product that has not been melted in the container floats in the liquid, and thus, is heated in the upper portion of the container and to be melted intensively. Since such a phenomenon continuously occurs in the container, the melting of the entire bio-derived frozen product in the container is promoted. Therefore, it is possible to rapidly melt the bio-derived frozen product contained in the container. In addition, it is hygienic since hot water does not directly contact an outer surface of the container.

The melting device may include a cooling unit that cools a lower portion of the container supported in the vertically oriented state.

With this configuration, the bio-derived liquid product that has melted in the container and moved to the lower portion is cooled by the cooling unit, and thus, does not lose its activity. That is, it is possible to perform the melting that is gentle for the bio-derived substance.

In the above melting device, the container may have a flat shape, the main body may have a housing portion capable of housing the container, and the heating unit may have a first heating unit and a second heating unit respectively provided on wall portions of the housing portion that face each other.

With this arrangement, it is possible to effectively heat the upper portion of the container supported in the vertically oriented state.

In the above melting device, the main body may have a housing portion capable of housing the container, and the container may have an opening that is open upward.

With this arrangement, it is easy to insert and remove the container from the main body.

Another aspect of the present disclosure is a melting method for melting a bio-derived frozen product contained in a container, and includes: supporting the container containing the bio-derived frozen product in a vertically oriented state; and heating only an upper portion of the container supported in the vertically oriented state.

The melting method may further include cooling a lower portion of the container supported in the vertically oriented state.

According to the melting device and the melting method of the present disclosure, it is possible to hygienically and rapidly melt the bio-derived frozen product contained in the container.

### BRIEF DESCRIPTION OF THE DRAWINGS

FIG. 1 is a perspective view of a melting device according to a first embodiment of the present disclosure;

FIG. 2 is a schematic cross-sectional view of the melting device (before melting a bio-derived frozen product) according to the first embodiment of the present disclosure;

FIG. 3 is a schematic cross-sectional view of the melting device (during melting of the bio-derived frozen product) according to the first embodiment of the present disclosure; and

FIG. 4 is a schematic cross-sectional view of a melting device according to a second embodiment of the present disclosure.

### DETAILED DESCRIPTION

Hereinafter, embodiments of a melting device and a melting method according to the present disclosure will be described with reference to the accompanying drawings.

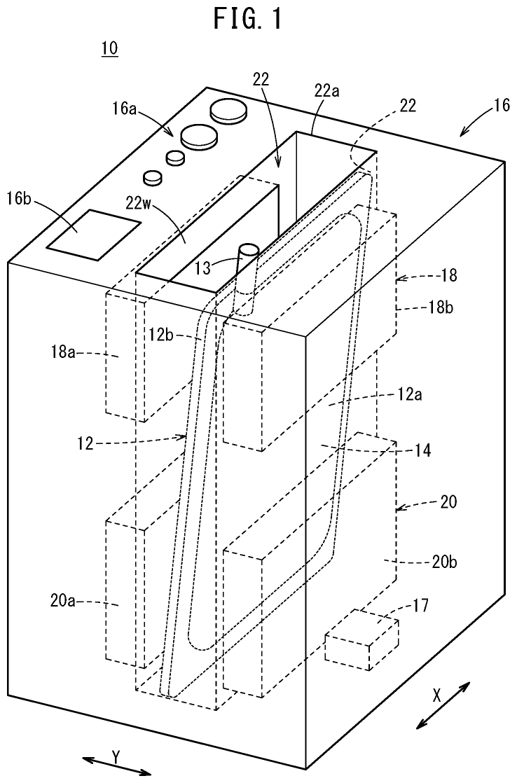

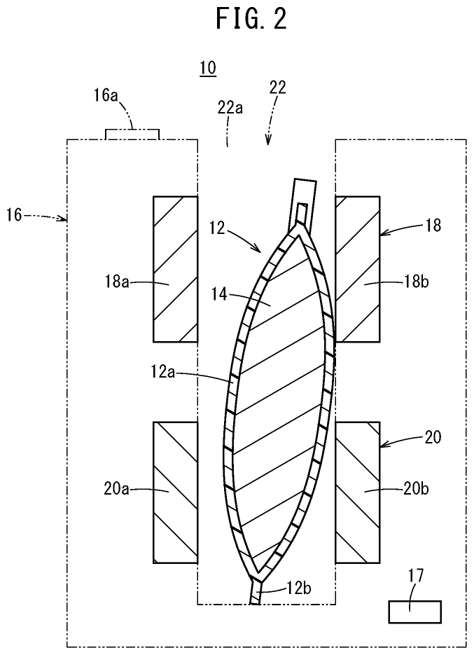

A melting device 10 according to a first embodiment illustrated in FIGS. 1 and 2 is used to melt a bio-derived frozen product 14 contained in a container 12. The container 12 is, for example, a bag-shaped flat soft bag formed of a resin film. The container 12 has a bag-shaped container body 12a having a storage chamber for the bio-derived frozen product 14, and a circumferential edge portion 12b surrounding an outer circumferential portion of the container body 12a. A port 13 configured to extract a bio-derived liquid product 14L from the inside of the container 12 is provided in the circumferential edge portion 12b of the container 12.

The bio-derived frozen product 14 contained in the container 12 is obtained by freezing a liquid containing a bio-derived substance (bio-derived liquid product 14L). Examples of the bio-derived liquid product 14L include a cell suspension, blood, plasma, and the like. Examples of the cell suspension may include hematopoietic stem cells (umbilical cord blood, bone marrow fluid, a peripheral blood stem cell, and the like) which may be used for stem cell transplantation. Cells in the cell suspension are not limited thereto, and are cells such as adherents cells such as myoblasts, cardiomyocytes, fibroblasts, synovial cells, epithelial cells, endothelial cells, hepatocytes, pancreatic cells, renal cells, adrenal cells, periodontal ligament cells, gingival cells, periosteal cells, skin cells, and chondrocytes, blood cells and blood components such as whole blood, red blood cells, white blood cells, lymphocytes (T lymphocytes, B lymphocytes), dendritic cells, plasma, platelets, and platelet-rich plasma, Bone Marrow Derived Mononuclear Cells, hematopoietic stem cells, ES cells, pluripotent stem cells, iPS cell-derived cells (for example, iPS cell-derived cardiomyocytes), mesenchymal stem cells (for example, those derived from bone marrow, adipose tissue, peripheral blood, skin, hair root, muscle tissue, endometrium, placenta, cord blood, and the like), and/or gametes (e.g., sperm cells and/or egg cells). These cells may be cells into which a gene used for gene therapy or the like has been introduced.

As illustrated in FIG. 1, the melting device 10 includes a main body 16, a heating unit 18 provided in the main body 16, and a cooling unit 20 provided in the main body 16.

The main body 16 is configured to support the container 12 in a vertically oriented state. Regarding the container 12, the “vertically oriented state” includes not only a case where the longitudinal axis of the container 12 is parallel to the vertical direction but also a state of being inclined at a certain angle (e.g., an angle within 45°) with respect to the vertical direction as illustrated in FIGS. 1 and 2. It is preferable that the main body 16 be capable of supporting the container 12 such that the angle of the longitudinal axis of the container 12 with respect to the vertical direction (e.g., a gravity direction, etc.) falls within a 0° to 30° angle range.

Specifically, the main body 16 has a housing portion 22 that can house the container 12 in the vertically oriented state. The housing portion 22 is formed in a flat shape (e.g., as a rectangular, or other shaped, slot) that extends in the vertical direction and has a thickness in the horizontal direction. That is, a horizontal cross-sectional shape of the housing portion 22 has a shape in which a dimension (e.g., a length) in a first direction (direction of an arrow X) is relatively large and a dimension (e.g., a width) in a direction perpendicular to the first direction (direction of an arrow Y) is relatively small. for example, the length may be twice the size of the width. The housing portion 22 has an opening 22a that is open on an upper surface of the main body 16.

The main body 16 is provided with: an operation section 16a including an operation button configured to operate operation start, operation stop, and the like, and setting buttons for various settings; and a display 16b that displays various types of information (e.g., set time, remaining time, set temperature, and the like). The main body 16 may be provided with a lid that closes the opening 22a.

The heating unit 18 heats only an upper portion of the container 12 supported in the vertically oriented state. The “upper portion of the container 12” indicates, for example, a region above a central position in the vertical direction of the container body 12a of the container 12 supported in the vertically oriented state, or a region located at the uppermost part among regions obtained by vertically dividing the container body 12a of the container 12 supported in the vertically oriented state into three equal parts. The “upper portion of the container 12” may be a region narrower in the vertical direction than the above-described region.

The heating unit 18 is, for example, a heater (e.g., a sheath heater, a cartridge heater, a flat heater, and the like). In the first embodiment, the heating unit 18 includes a first heating unit 18a and a second heating unit 18b that are provided on upper portions of wall portions 22w of the housing portion 22 that face each other. The heating unit 18 is controlled by a control unit 17 provided in the main body 16. For instance, the heating unit 18 may supply heat to a portion, or specific region, inside the housing portion 22 (e.g., an upper portion of the cavity of the housing portion 22, etc.) based on a control signal received from the control unit 17. In some embodiments, the heating unit 18 may be provided on only one of the wall portions 22w of the housing portion 22 that face each other.

The cooling unit 20 cools a lower portion of the container 12 supported in the vertically oriented state. The “lower portion of the container 12” indicates, for example, a region below the central position in the vertical direction of the container body 12a of the container 12 supported in the vertically oriented state, or a region located at the lowermost part among regions obtained by vertically dividing the container body 12a of the container 12 supported in the vertically oriented state into three equal parts. The “lower portion of the container 12” may be a region narrower in the vertical direction than the above-described region.

The cooling unit 20 is arranged below the heating unit 18. The cooling unit 20 is, for example, a cooling module using a Peltier element (e.g., a thermoelectric cooler, etc.). In the first embodiment, the cooling unit 20 has a first cooling unit 20a and a second cooling unit 20b that are provided on lower portions of the wall portions 22w of the housing portion 22 that face each other. The cooling unit 20 is controlled by the control unit 17 provided in the main body 16. In some embodiments, the cooling unit 20 may be provided only on one of the wall portions 22w of the housing portion 22 that face each other. The cooling unit 20 may be a cooling module that uses a gas refrigerant.

Next, an operation of the melting device 10 arranged as described above will be described.

When the bio-derived frozen product 14 contained in the container 12 is ready to be melted, or otherwise thawed, using the melting device 10, the container 12 in which the bio-derived frozen product 14 is stored is inserted into the housing portion 22 of the main body 16 through the opening 22a as illustrated in FIG. 2. As a result, the container 12 is housed in the melting device 10.

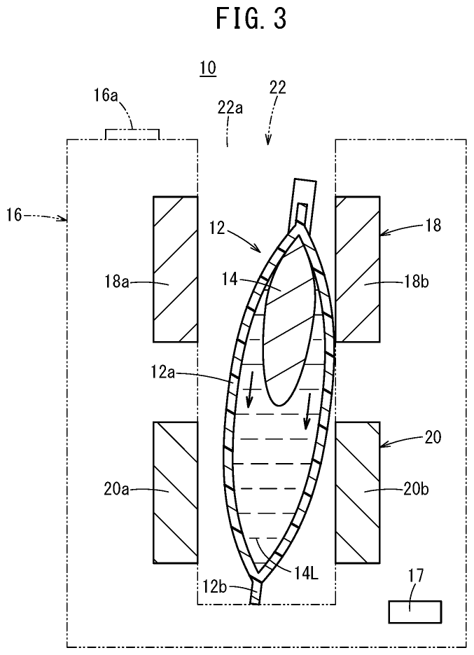

When a start button of the operation section 16a (FIG. 1) provided in the main body 16 is operated, the melting device 10 starts operating. Specifically, as the heating unit 18 (e.g., heater, heating element, etc.) increases in temperature, the bio-derived frozen product 14 in the upper part of the container 12 facing the heating unit 18 is heated, the heated portion melts to form the bio-derived liquid product 14L, as illustrated in FIG. 3. In FIG. 3, the bio-derived liquid product 14L melted in the upper portion of the container 12 flows to the lower portion of the container 12 due to the specific gravity. The bio-derived frozen product 14 that has not been melted in the container 12 floats in the bio-derived liquid product 14L, and thus, is heated in the upper portion of the container 12 to be melted. Such a phenomenon continuously occurs in the container 12, and the entire bio-derived frozen product 14 is eventually melted, or completely thawed.

In parallel with the heating by the heating unit 18, the cooling unit 20 cools the lower portion of the container 12. As a result, the bio-derived liquid product 14L that has melted and moved to the lower portion of the container 12 is cooled. In this case, the cooling unit 20 cools the lower portion of the container 12 to a temperature suitable for holding the activity of the bio-derived liquid product 14L without freezing the bio-derived liquid product 14L (e.g., 2° C. to 6° C., and in some embodiments about 4° C. (3.5° C. to 4.4° C.), etc.).

The melting device 10 according to the first embodiment has the following effects.

As described above, the liquid (bio-derived liquid product 14L) obtained as the bio-derived frozen product 14 is melted in the upper portion of the container 12 flows to the lower portion of the container 12 according to the melting device 10. The bio-derived frozen product 14 that has not been melted in the container 12 floats in the bio-derived liquid product 14L, and thus, is heated in the upper portion of the container 12 to be melted. Since such a phenomenon continuously occurs (e.g., as a cycle, etc.) in the container 12, the melting of the entire bio-derived frozen product 14 in the container 12 is promoted. Therefore, it is possible to rapidly melt the bio-derived frozen product 14 contained in the container 12. In addition, it is hygienic since hot water does not directly contact an outer surface of the container 12.

The melting device 10 includes the cooling unit 20 that cools the lower portion of the container 12 supported in the vertically oriented state. With this configuration, the bio-derived liquid product 14L that has melted in the container 12 and moved to the lower portion is cooled by the cooling unit 20, and thus, does not lose its activity. That is, it is possible to perform the melting that is gentle for the bio-derived substance.

The container 12 has the flat shape. The main body 16 has the housing portion 22 that houses the container 12. Further, the heating unit 18 includes the first heating unit 18a and the second heating unit 18b that are provided on the wall portions 22w of the housing portion 22 that face each other. With this arrangement, it is possible to effectively heat the upper portion of the container 12 supported in the vertically oriented state.

In some embodiments, the cooling unit 20 may not be necessary and may be excluded from the melting device 10. Even if the cooling unit 20 is not provided, or is excluded from the melting device 10, the bio-derived frozen product 14 cools the bio-derived liquid product 14L while the unmelted bio-derived frozen product 14 remains in the container 12. Therefore, the cooling effect can be obtained to some extent.

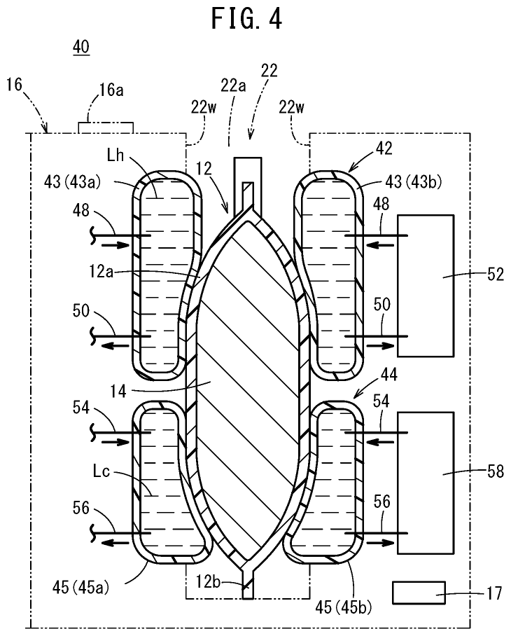

A melting device 40 according to a second embodiment illustrated in FIG. 4 may be different from the melting device 10 according to the first embodiment in that the melting device 40 according to the second embodiment may include a heating unit 42 and a cooling unit 44 each in the form of a bag.

For instance, the heating unit 42 may have a heating bag 43 filled with a heating liquid Lh. The heating bag 43 may be arranged to come into contact with an upper outer surface of the container 12 in the state of being housed in the housing portion 22. The heating bag 43 may comprise a soft bag that is made of, for example, a resin film, or other material, that is easily deformed. As illustrated in FIG. 4, the heating unit 42 has a first heating bag 43a and a second heating bag 43b that are respectively provided on the wall portions 22w of the housing portion 22 of the main body 16 that face each other. The container 12, which contains the bio-derived frozen product 14 and is arranged to be vertically oriented in the housing portion 22 of the main body 16, is sandwiched by the first heating bag 43a and the second heating bag 43b.

The main body 16 is provided with a heating circulation unit 52 that heats the liquid Lh to a predetermined temperature (e.g., 30° C. to 40° C.) to supply the liquid Lh to the first heating bag 43a and the second heating bag 43b via an introduction line 48, and sucks, or removes, the liquid Lh from the first heating bag 43a and the second heating bag 43b via an extraction line 50 to heat the liquid Lh again (e.g., in a continuous heating and reheating cycle, etc.).

The cooling unit 44 has a cooling bag 45 filled with a cooling liquid Lc. The cooling bag 45 is arranged to come into contact with a lower outer surface of the container 12 in the state of being housed in the housing portion 22. The cooling bag 45 may comprise a soft bag that is made of, for example, a resin film, or other material, that is easily deformed. The cooling unit 44 has a first cooling bag 45a and a second cooling bag 45b that are respectively provided on the wall portions 22w of the housing portion 22 of the main body 16 that face each other. The container 12, which contains the bio-derived frozen product 14 and is arranged to be vertically oriented in the housing portion 22 of the main body 16, is sandwiched by the first cooling bag 45a and the second cooling bag 45b.

The main body 16 is provided with a cooling circulation unit 58 that cools the liquid Lc to a predetermined temperature (e.g., 2° C. to 6° C.) to supply the liquid Lc to the first cooling bag 45a and the second cooling bag 45b via an introduction line 54, and sucks, or removes, the liquid Lc from the first cooling bag 45a and the second cooling bag 45b via an extraction line 56 to cool the liquid Lc again (e.g., in a continuous cooling and re-cooling cycle, etc.).

It is an aspect of the melting device 40 according to the second embodiment arranged in this manner, to hygienically and rapidly melt the bio-derived frozen product 14 contained in the container 12 and to perform melting that is gentle for the bio-derived substance, which is similar to the melting device 10 according to the first embodiment. In some embodiments, the cooling unit 44 may not be necessary and may be excluded from the melting device 40.

A melting device according to an aspect of the present disclosure may include the heating unit 18 illustrated in FIG. 2 and the cooling unit 44 illustrated in FIG. 4. A melting device according to an aspect of the present disclosure may include the heating unit 42 illustrated in FIG. 4 and the cooling unit 20 illustrated in FIG. 2.

The present disclosure is not limited to the above-described embodiment, and various modifications can be made within a scope not departing from a gist of the present disclosure.

What is claimed is:

1. A melting device that melts a bio-derived frozen product contained in a container, the melting device comprising:
a main body capable of supporting the container in a vertically oriented state; and
a heating unit that heats only an upper portion of the container when the container is supported in the vertically oriented state.

2. The melting device of claim 1, further comprising:
a cooling unit that cools a lower portion of the container when the container is supported in the vertically oriented state.

3. The melting device of claim 1, wherein
the container has a flat shape,
wherein the main body has a housing portion capable of housing the container, and
wherein the heating unit has a first heating unit and a second heating unit respectively provided on wall portions of the housing portion that face each other.

4. The melting device of claim 3, wherein
the housing portion has an opening disposed on a first end of the melting device.

5. The melting device of claim 4, wherein the first heating unit and the second heating unit are deformable, and wherein the first heating unit and the second heating unit at least partially conform to a curvature of the container after the container is inserted into the first end of the housing portion.

6. The melting device of claim 1, wherein the melting device further comprises:
a heating circulation unit, wherein the heating circulation unit is in fluidic communication with the heating unit through an introduction line and an extraction line, wherein the introduction line introduces a fluid in a heated state to the heating unit, and wherein the extraction line removes the fluid from the heating unit.

7. The melting device of claim 1, further comprising:
a cooling unit that cools a lower portion of the container when the container is supported in the vertically oriented state, wherein the cooling unit comprises a first cooling unit and a second cooling unit, wherein the first cooling unit is disposed on a first wall of the housing, wherein the second cooling unit is disposed on a second wall of the housing, and wherein first wall and the second wall face one another.

8. The melting device of claim 7, wherein the first cooling unit and the second cooling unit are flexible, and wherein the first cooling unit and the second cooling unit at least partially conform to a curvature of the container when the container is inserted into the first end of the housing portion.

9. A melting method for melting a bio-derived frozen product contained in a container, the melting method comprising:
supporting the container containing the bio-derived frozen product in a vertically oriented state; and
heating only an upper portion of the container supported in the vertically oriented state.

10. The melting method of claim 9, further comprising:
cooling a lower portion of the container supported in the vertically oriented state.

11. A system for melting a bio-derived frozen product contained in a container, the system comprising:
a control unit; and
a melting device, the melting device comprising:
a main body containing a cavity that receives and maintains the container in a vertically oriented state; and
a heating unit disposed in an upper portion of the cavity of the main body and operatively connected to the control unit, wherein the heating unit supplies heat to the container based on a control signal received from the control unit.

12. The system of claim 11, wherein the heating unit further comprises:
a first heating unit; and
a second heating unit, wherein the first heating unit is disposed on a first cavity wall of the cavity, wherein the second heating unit is disposed on a second cavity wall of the cavity, and wherein the container in the vertically oriented state contacts at least one of the first heating unit and the second heating unit.

13. The system of claim 12, wherein the container contacts the first heating unit and does not contact the second heating unit.

14. The system of claim 12, wherein an angle of a longitudinal axis of the container in the vertically oriented state relative to a vertical direction falls within a 0° to 30° angle range.

15. The system of claim 11, wherein the melting device further comprises:
a cooling unit, wherein the cooling unit is disposed in a lower portion of the cavity of the main body and configured to remove heat from the container.

16. The system of claim 15, wherein the cooling unit comprises a first cooling unit and a second cooling unit, wherein the first cooling unit is disposed on a first cavity wall, wherein the second cooling unit is disposed on a second cavity wall.

17. The system of claim 11, wherein the melting device further comprises:
a heating circulation unit, wherein the heating circulation unit is in fluidic communication with the heating unit through an introduction line and an extraction line, wherein the heating circulation unit introduces a liquid in a heated state to the heating unit via the introduction line, and wherein the heating circulation unit extracts the liquid in an unheated state from the heating unit via the extraction line.

18. The system of claim 17, wherein the heating circulation unit is operatively connected to the control unit.

19. The system of claim 11, wherein the cavity is shaped as a rectangular slot having a cross-section including a width and a length extending a depth into the main body.

20. The system of claim 11, wherein the melting device further comprises:
a cooling unit, wherein the cooling unit is disposed in a lower portion of the cavity of the main body and configured to remove heat from a lower portion of the container; and
a cooling circulation unit, wherein the cooling circulation unit is in fluidic communication with the cooling unit through an introduction line and an extraction line, wherein the cooling circulation unit introduces a liquid in a cooled state to the cooling unit via the introduction line, and wherein the cooling circulation unit extracts the liquid from the heating unit via the extraction line.
